(19) United States
(12) Patent Application Publication
(10) Pub. No.: US 2022/0183942 A1
(43) Pub. Date: Jun. 16, 2022

(54) CRACKLING HAND SANITIZER FORMULATIONS AND ASSOCIATED METHODS

(71) Applicant: Rubbermaid Commercial Products LLC, Atlanta, GA (US)

(72) Inventors: Nirali Patel, Winchester, VA (US); Carlo Jeunen, St-Truiden (BE)

(21) Appl. No.: 17/545,588

(22) Filed: Dec. 8, 2021

Related U.S. Application Data
(60) Provisional application No. 63/125,141, filed on Dec. 14, 2020.

(51) Int. Cl. A61K 8/31; A61Q 17/00

(52) U.S. Cl. CPC A61K 8/31; A61Q 17/005

(57) ABSTRACT

A composition and associated methods of manufacture for a new type of hand sanitizer that has a “crackling” effect when the user rubs it between their hands. Crackling hand sanitizer formulations promote hand sanitizing because of the interactive user experience provided during application and use of the formulations. A hand sanitizer concentrate solution mixed with a propellant and thickening agent results in a formulation that provides a crackling effect when applied to the hands by a user. The “crackling effect” is a physical reaction when the formulation is in contact with the user's skin, in which the propellant evaporates, causing a crackling sound while also providing a tingling or effervescent feeling on the skin.

### CROSS-REFERENCE TO RELATED APPLICATION

This application claims the benefit of U.S. Provisional Application No. 63/125,141 filed Dec. 14, 2020, which is incorporated by reference herein in its entirety.

### FIELD OF THE DISCLOSURE

The present disclosure relates to a composition and associated methods of manufacture for a new type of hand sanitizer that has a “crackling” effect when the user rubs it between their hands.

### BACKGROUND

Hand sanitizer formulations are known that allow for reducing bacteria and infectious agents on a user's skin without the need for soap and water. Hand sanitizer formulations beneficially offer an alternative to washing with soap and water, particularly in situations where soap and water may not be available.

Hand sanitizer formulations typically are provided in the form of liquid, gel, or foam compositions that are applied to the hands. Such compositions may be rubbed on the skin and then allowed to dry. Because many hand sanitizers contain relatively high amounts of alcohol, users may experience skin drying after use.

There is a need for improved hand sanitizing formulations that reduce the effect of skin drying and promote continued use.

### DETAILED DESCRIPTION

The crackling hand sanitizer formulations of the present disclosure promote hand sanitizing because of the interactive user experience provided during application and use of the formulations. Specifically, it has been discovered that a hand sanitizer concentrate solution mixed with a propellant and thickening agent results in a formulation that provides a crackling effect when applied to the hands by a user. As used herein, the phrase “crackling effect” refers to a physical reaction when the formulation contact with the user's skin (e.g., through hand rubbing), in which the propellant evaporates thereby causing a crackling sound while also providing a tingling or effervescent feeling on the skin.

For example, in the formulations described herein, the propellant may be captured and partially dissolved in a specially developed gel, to control the amount of foam formation and retain a gel-like quality that captures the propellant in suspension. When applied to the skin by a. user, the propellant is released from the thickening agent/gel through the action of application or rubbing hands together. The result is the propellant being released from the gel, which provides the crackling effect.

Formulations

In certain embodiments, the crackling hand sanitizer composition contains at least an alcohol, a thickening agent, and a propellant, wherein the propellant is suspended in the thickening agent, such that upon rubbing contact in a user's hands, the propellant evaporates from the thickening agent, causing the physical crackling effect.

The alcohol may be any suitable alcohol, such as lower hydrocarbon chain alcohols, like C₁-C₄ alcohols. In certain embodiments, the alcohol is ethanol. The alcohol may act as a carrier and a solvent within the composition and may be present in an amount of about 10 to about 20 percent, by weight of the composition. As used herein, the term “about” when used with

reference to a numerical value, refers to an amount that is plus or minus up to 3 percent of the stated numerical value. Beneficially, the compositions described herein may have a relatively low alcohol content, thereby minimizing the skin drying effect that may be experienced with traditional hand sanitizer formulations containing significantly higher alcohol content.

The compositions also contain a thickening agent that acts as the suspension agent for the propellant. The concentration and type of thickening age y upon specific demands. In some embodiments, the thickening agent is hydroxyethyl cellulose. However, other types of cellulose thickening agents with proven solubility in water may be used. In certain embodiments, the thickening agent is present in an amount of about 0.01 to about 1 percent, by weight of the composition.

In use, the thickening agent may capture the propellant upon release from a pressurized dispensing container. The propellant will be released from the gel when mechanical action (e.g., hand rubbing) takes place. Releasing the propellant from the gel results in a crackling sound and tingling feeling on the skin, which helps to enhance the user experience and thereby promotes repeated use of the sanitizer and improves hygiene.

The compositions also contain a suitable propellant. For example, the propellant may be butane and/or Liquefied Petroleum Gas (LPG). In some embodiments, the propellant is present in an amount of about 25 to about 65 percent, by weight of the composition; however, type and concentration ratio of propellant can vary depending on the required output and appearance of the formulation. Examples of suitable ratios are described in the Examples.

In some embodiments, the composition also contains an active antibacterial agent or biocide to provide further sanitizing effect of the formulation. In certain embodiments, the biocide includes a quaternary ammonium compound, such as benzalkonium chloride. For example, the biocide may be present in an amount of about 0.01 to about 1 percent, by weight of the composition, such as from about 0.05 to about 0.015 percent, by weight of the composition.

In some embodiments, the composition also contains an emulsifier that act as multifunctional ingredient to provide good emulsification and emolliency to the resulting formulation. The concentration and type can vary upon specific demands. In certain embodiments, the emulsifier includes PPG-5 Ceteth-20, PEG-40 Hydrogenated Castor Oil, or both. In certain embodiments, the emulsifier is present in an amount of about 0.01 to about 2 percent, by weight of the composition, such as from about 0.20 to about 0.70 percent, by weight of the composition.

In some embodiments, the composition also contains a humectant or cushioning agent, which provides a soft, cushioned feel on the skin while also acting as a moisturizer and preventing drying of the skin. The concentration and type of humectant can vary upon specific demands. In certain embodiments, the humectant is dimethicone, propanediol, or both. For example, the humectant, be present in an amount of about 0.01 to about 2 percent, by weight of the composition, such as from about 0.7 to about 0.85 percent, by weight of the composition. For example, a humectant such as dimethicone may also provide defoaming properties to the formulation. By breaking the foam, the desired crackling effect may be obtained when the propellant breaks free from the gel.

In some embodiments, the composition also includes a fragrance to provide a scented formulation. The fragrance may be any suitable chemistry, including essential oils and the like. For example, the fragrance may be present in an amount of about 0.01 to about 1 percent, by weight of the composition, such as from about 0.05 to about 0.25 percent, by weight of the composition.

In some embodiments, the composition further includes one or more additional solvents, such as water. In certain embodiments, the composition also includes sorbitol as a solvent.

Thus, the crackling hand sanitizer formulations described herein may include a hand sanitizer concentrate mixed with a propellant and thickening agent that provides a crackling effect when applied by a user. The propellant may be captured and partially dissolved in a gel formed from the thickening agent, optionally with an emulsifier and/or humectant. The propellant and thickening agent gel play be combined with alcohol and to form the hand sanitizing formulation. For example, in certain embodiments, the alcohol is ethanol, present in an amount of about 10 to about 20 percent, by weight of the composition, the thickening agent is hydroxyethyl cellulose, present in an amount of about 0.1 to about 0.8 percent, by weight of the composition, and the propellant is butane, present in an amount of about 45 to about 65 percent, by weight of the composition. The formulation may also include further additives, as described above.

It was discovered that the presently described compositions can be formulated to provide the aforementioned crackling effect, which can be further customized by the choice of fragrance and thickener in the formulation. The crackling effect provides an interactive user experience that is unique to the market, and which promotes the frequent use of hand sanitizer. Additionally, the present formulations provide a soft skin feel experience and minimize drying of the skin, as compared to traditional hand sanitizer formulations.

Methods

Methods of manufacturing the crackling hand sanitizer formulations described herein are also provided. In certain embodiments, the method includes combining the alcohol with the thickening agent to form a hand sanitizer concentrate, and combining the hand sanitizer concentrate with the propellant. In some embodiments, the thickening agent is combined with water prior to combination with the alcohol. In some embodiments, the method includes forming an alcohol premix by combining the alcohol with one or more of: a biocide, an emollient, a humectant, and a fragrance, prior to combination of the alcohol with the thickening agent. For example, the hand sanitizer concentrate may be mixed with the propellant in a ratio of from 35:65 to 75:25.

Dispensers

Dispensers containing the crackling hand sanitizer formulations described herein are also provided. For example, the crackling hand sanitizer formulation may be provided in a suitable pressurized dispenser.

In certain embodiments, the crackling hand sanitizer formulation is provided in a pressurized container, such as a can, having a suitable valve dispenser, whereby the valve has an effect on the output appearance of the gel and the output quantity. For example, suitable types of valves to use include, but are not limited to the following: mousse valve, tilted valve, metered valve, bag on valve (BOV).

The actuator used in the dispenser may also have an effect on the output of the gel. Proper choice of actuator ill prevent post-foaming in the actuator and dripping of the gel from the refill/dispenser.

Depending on the diameter and the shape of the nozzle and nozzle opening, the actuator skirt dimension, and the position of the actuator hinge or rotation point, the output of the hand sanitizer gel can be affected.

Additionally, balls can be added to the pressurized dispenser containing the gel and propellant to ensure proper mixing/homogenizing of the propellant into the liquid until the required gel/propellant phase is established. These balls are commonly used in for example spray paint aerosol cans; however, have not been employed in cosmetic and drug products to our knowledge.

Examples

Various embodiments of the crackling hand sanitizer formulations described herein were manufactured and tested.

First, by selecting different ratios of gel and propellant, it was found that the output composition can be influenced varying from foam to gel. In addition, the intensity of the crackling effect was found to be tailorable by varying the gel to propellant ratio.

A preferred list of different propellants that were tested and the composition ratios found to be suitable for each are as follows:

| Propellant type | Propellant | Gel |
|---|---|---|
| Butane | 25-65% | 75-35% |
| LPG | 25-50% | 75-50% |

Next, various crackling hand sanitizer formulations were prepared and tested. A first formulation was prepare as follows:

| | | % w/w | | | |
|---|---|---|---|---|---|
| | % | (raw | | | |
| INCI name | w/w | material) | Function | Trade name | Supplier |
| Butane | 55 | 55 | Propellant | | |
| Water (Aqua) | 27 | 27 | Solvent | Demineralized | |
| | | | | Tap Water | |
| Ethanol 99.8% | 15 | 15 | Solvent | | |
| PPG-5 | 0.65 | 0.65 | Emollient | Procetyl | Croda |
| Ceteth-20 | | | | AWS- | |
| | | | | LQ-(RB) | |
| Benzalkonium | 0.13 | 0.26 | Antimicrobial | Barquat DM- | Lonza |
| Chloride | | | Agent | 50 EP | |
| Water | 0.13 | | Solvent | | |
| PEG-40 | 0.24 | 0.24 | Emulsifier | Cremaphor | BASF |
| Hydrogenated | | | | CO40 | |
| Castor Oil | | | | | |
| Sorbitol | 0.6 | 0.6 | Solvent | Sorbitol 70% | |
| | | | | NC | |
| Hydroxyethylcellulose | 0.3 | 0.3 | Thickener | Natrosol | Ashland |
| | | | | 250HHR | |
| Dimethicone | 0.8 | 0.8 | Cushioning | Mirasil | Elkem |
| | | | agent | DM-50 | |
| Fragrance | 0.15 | 0.15 | Fragrance | Purification | Drom |
| | | | Ingredient | 49406159 | |
| | | 100 | | | |

The above formulation was found to provide a superior feel, softness, and crackling effect when applied by a user.

Next, another formulation was prepared, similar to the above formulation, but containing propanediol as the cushioning agent instead of dimethicone. Again, the formulation provided the described hand feel and crackling effect when applied by a user.

| | | % w/w | | | |
|---|---|---|---|---|---|
| | % | (raw | | | |
| INCI name | w/w | material) | Function | Trade name | Supplier |
| Butane | 55 | 55 | Propellant | | |
| Water (Aqua) | 27.6 | 27.6 | Solvent | Demineralized | |
| | | | | Tap Water | |
| Ethanol 99.8% | 15 | 15 | Solvent | | |
| PPG-5 | 0.65 | 0.65 | Emollient/ | Procetyl | Croda |
| Ceteth-20 | | | Emulsifier | AWS- | |
| | | | | LQ-(RB) | |
| Benzalkonium | 0.13 | 0.26 | Antimicrobial | Barquat DM- | Lonza |
| Chloride | | | Agent | 50 EP | |
| Water | 0.13 | | Solvent | | |
| PEG-40 | 0.24 | 0.24 | Emulsifier | Cremaphor | BASF |
| Hydrogenated | | | | CO40 | |
| Castor Oil | | | | | |
| Sorbitol | 0.6 | 0.6 | Solvent | Sorbitol 70% | |
| | | | | NC | |
| Hydroxyethylcellulose | 0.3 | 0.3 | Thickener | Natrosol | Ashland |
| | | | | 250HHR | |
| Propanediol | 0.2 | 0.2 | Cushioning | Zemea | Dupont |
| | | | agent | Propanediol | Tate & |
| | | | | | Lyle |
| Fragrance | 0.15 | 0.15 | Fragrance | Purification | Drom |
| | | | Ingredient | 49406159 | |
| | | 100 | | | |

Suitable methods for manufacturing crackling hand sanitizer formulations to achieve the desired results were also tested.

In one manufacturing method, the demineralized water was heated to 45° C. and the hydroxyethyl cellulose was added slowly and carefully while stirring, letting it swell. The sorbitol was added while stirring until dissolved. The benzalkonium chloride, emollients/emulsifiers, humectant, and fragrance were combined to the ethanol to form a premix. The ethanol premix was added to the batch and the ingredients were mixed until homogeneous. To fill the dispenser, the sanitizer concentrate was combined with the propellant in a weight ratio of 55:45. The result was a formulation having the appearance of a viscous, turbid liquid with specific gravity of: 0.95 g/ml +/− 0.006 and a pH of 6.0 +/− 0.2.

Other manufacturing methods may be used depending on the ingredients of the formulation, and the mixing equipment available. For example, certain ingredients may be added to the batch mixture directed instead of to the ethanol premix. It should be understood that the above-described method is provided merely for exemplary purposes and other orders of steps and combinations of the ingredients may be used to achieve suitable cracking hand sanitizer formulations in accordance with this disclosure.

While the disclosure has been described with reference to a number of embodiments, it will be understood by those skilled in the art that the invention is not limited to such disclosed embodiments. Rather, the invention can be modified to incorporate any number of variations, alterations, substitutions, or equivalent arrangements not described herein, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A crackling hand sanitizer composition comprising:
an alcohol;
a thickening agent; and
a propellant,
wherein the propellant is suspended in the thickening agent, such that upon rubbing contact in a user's hands, the propellant evaporates from the thickening agent, causing a physical crackling effect.

2. The composition of claim 1, wherein the alcohol is ethanol.

3. The composition of claim 1, wherein the alcohol is present in an amount of about 10 to about 20 percent, by weight of the composition.

4. The composition of claim 1, wherein the thickening agent s hydroxyethyl cellulose.

5. The composition of claim 1, wherein the thickening agent is present in an amount of about 0.01 to about 1 percent, by weight of the composition.

6. The composition of claim 1, wherein the propellant is butane or Liquefied Petroleum Gas (LPG).

7. The composition of claim 1, w herein the propellant is present in an amount of about 25 to about 65 percent, by weight of the composition.

8. The composition of claim 1, further comprising a biocide.

9. The composition of claim 8, wherein the biocide comprises a quaternary ammonium compound.

10. The composition of claim 8, the biocide is benzalkonium chloride.

11. The composition of claim 1, further comprising an emulsifier

12. The composition of claim 11, wherein the emulsifier comprises one or more of PPG-5 Ceteth-20 and PEG-40 Hydrogenated Castor Oil.

13. The composition of claim 1, further comprising a humectant.

14. The composition of claim 13, wherein the humectant comprises one or more of dimethicone and propanediol.

15. The composition of claim 1, further comprising a fragrance.

16. The composition of claim 1, further comprising a solvent, the solvent being one or more of water and sorbitol.

17. A hand sanitizer dispenser, comprising:
a container housing the composition comprising:
an alcohol;
a thickening agent; and
a propellant,
wherein the propellant is suspended in the thickening agent,
wherein the container comprises a valve for selectively dispensing the composition from the container, and
wherein the container is pressurized.

18. A method of making the composition, the composition comprising:
an alcohol;
a thickening agent; and
a propellant,
wherein the propellant is suspended in the thickening agent, the method comprising:
combining the alcohol with the thickening agent to form a hand sanitizer concentrate; and
combining the hand sanitizer concentrate with the propellant.

19. The method of claim 18, wherein the thickening agent is combined with water prior to combination with the alcohol.

20. The method of claim 19, further comprising forming an alcohol premix by combining the alcohol with one or more of: a biocide, an emollient, a humectant, and a fragrance.
